(19) United States
(12) Patent Application Publication
(10) Pub. No.: US 2024/0000510 A1
(43) Pub. Date: Jan. 4, 2024

(54) AUTOMATIC GENERATION OF EDUCATIONAL CONTENT FROM USAGE OF OPTIMAL/POOREST USER

(71) Applicant: KONINKLIJKE PHILIPS N.V., EINDHOVEN (NL)

(72) Inventors: Paul Anthony SHRUBSOLE, ARNHEM (NL); Alexander Sebastian FURNICA, EINDHOVEN (NL)

(21) Appl. No.: 18/037,789

(22) PCT Filed: Nov. 18, 2021
(86) PCT No.: PCT/EP2021/082193

§ 371 Date: May 19, 2023

Related U.S. Application Data
(60) Provisional application No. 63/116,956, filed on Nov. 23, 2020.

(51) Int. Cl. A61B 34/10; G16H 30/20; G06T 19/00

(52) U.S. Cl. CPC A61B 34/10; G16H 30/20; G06T 19/006; A61B 2034/101

(57) ABSTRACT

A non-transitory computer readable medium stores a database (30) storing log data automatically generated by one or more medical devices (12); and instructions readable and executable by at least one electronic processor (16) to perform a method (100) for generating educational content units (38). The method includes: analyzing the log data (32) contained in the database to identify log data for an educational instance of a procedure performed using the one or more medical devices; creating a simulation of the identified educational instance of the procedure using the identified log data for the educational instance of the procedure; and generating an educational content unit (38) from the simulation.

### FIELD

The following relates generally to the medical arts, medical education arts, and medical device operations educational content generation arts, especially as directed to medical imaging devices.

### BACKGROUND

Complex medical devices offer great flexibility in how they can be used to diagnose, monitor, or treat patients. The performance of the medical device may depend strongly on how the operator uses the device, e.g. setting a non-optimal configuration may provide sub-optimal results whereas using a more optimal configuration may provide better results. Moreover, medical devices that are connected to the Internet or another electronic network may receive software or firmware upgrades over the network that provide new features or enhance existing features; however, these may be useless if the operator is not trained to effectively use the new or enhanced features. Thus, there is substantial benefit to offering education and support to get the best results from the medical devices according to the clinical needs of patients and according to the specializations, way of working of the staff, and the type of hospital or clinical practice. This further leads to a situation where it is difficult to provide all forms of educational content for all features of a device, especially when usage context of those features is being considered. In addition to this, there are early adopters who are educated about new or updated devices much earlier than other users. As a consequence, their usage patterns are more optimized and could be used as a benchmark or starting point for newer, less experienced users. However, there are many barriers to leveraging the usage patterns of early adopters for training other users, such as patient privacy and workflow efficiency considerations that can make recordation of a medical procedure infeasible.

Another issue with educational content for training on medical devices is that the training materials focus narrowly on the device itself. Often, standardized educational content is focused on explaining features of the medical device and their usage. However, there is rarely a focus on entire workflows being explained. For example, an Image Guided Therapy (IGT) procedure involves cooperative use of the IGT imaging device and its controller, a catheter, biopsy needle, or other interventional instrument and its controller, and possibly other components such as patient monitor devices, biopsy sample collection apparatus, and so forth. Educational content focusing on (for example) the interventional instrument and its controller may fail to provide useful training on the usage of this system with other systems such as the interventional instrument. Medical professionals need to perform procedures that utilize cooperating systems, yet these the training materials are usually narrowly focused on specific systems in isolation. It is also often not practical for exhaustive educational content for workflows to be created manually, as there would be too many possibilities in terms of features and enhancements to use within a particular workflow.

Even when workflow examples are provided, for example as part of the explanation of a particular feature, it is unlikely that it is relevant for all users. This “one-size-fits-all” approach to educational content creation leaves some users lacking confidence in adopting some features into their day-to-day work.

In addition, there are large experience gaps between users that are currently not dealt with or leveraged in any way. This way, often the knowledge gaps between users only grow larger.

The following discloses certain improvements to overcome these problems and others.

### SUMMARY

In one aspect, a non-transitory computer readable medium stores a database storing log data automatically generated by one or more medical devices; and instructions readable and executable by at least one electronic processor to perform a method for generating educational content units. The method includes: analyzing the log data contained in the database to identify log data for an educational instance of a procedure performed using the one or more medical devices; creating a simulation of the identified educational instance of the procedure using the identified log data for the educational instance of the procedure; and generating an educational content unit from the simulation.

In another aspect, an apparatus comprises a database storing log data automatically generated by one or more medical imaging devices; at least one electronic processor; and instructions readable and executable by the at least one electronic processor to perform a method for generating educational content units. The method includes: retrieving log data from the database for an instance of a medical imaging procedure performed using the one or more medical devices; simulating the instance of the medical imaging procedure using the retrieved log data to generate a simulation of the instance of the medical imaging procedure; and generating an educational content unit from the simulation of the instance of the medical imaging procedure.

In another aspect, a method for generating educational content units includes: retrieving log data from the database for an instance of a medical imaging procedure performed using the one or more medical devices; analyzing the log data contained in the database to identify log data for an educational instance of a procedure performed using the one or more medical devices; simulating the instance of the medical imaging procedure using the analyzed log data to generate a simulation of the instance of the medical imaging procedure; and generating an educational content unit from the simulation of the instance of the medical imaging procedure.

One advantage resides in providing educational content for training medical professionals for a medical procedure that utilizes a medical device or combination of medical devices, in which the educational content is generated from one or more actual instances of the medical procedure.

Another advantage resides in providing such educational content generated from one or more actual instances of the medical procedure, in which the instances may include positive examples in which the medical procedure was highly effective, negative examples in which some mistake may have been made, or combinations thereof.

Another advantage resides in providing such educational content generated from one or more actual instances of the medical procedure without recording video or audio of the instance(s) of the medical procedure, thereby obviating concerns about patient privacy and interference with the medical procedure.

Another advantage resides in generating educational content for operating a medical device from log data of the medical device.

Another advantage resides in generating educational content for a medical device tailored to individual technicians or groups of technicians who operate the device.

Another advantage resides in generating educational content for a medical device for a team of technicians who operate the medical device.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for generating educational content units in accordance with the present disclosure.

FIG. 2 shows exemplary flow chart operations of the system of FIG. 1.

### DETAILED DESCRIPTION

As used herein, a “medical procedure” refers to a medical workflow utilizing one or more medical devices to achieve a clinical purpose such as diagnosing a medical condition, providing a clinical therapy or treatment, screening for a specific illness, and/or so forth. Some examples of a medical procedure include, by way of non-limiting illustrative example: a medical imaging procedure utilizing a magnetic resonance imaging (MRI) scanner; an image guided therapy (IGT) procedure such as a catheterization procedure, biopsy procedure, or the like performed using an interventional instrument (e.g. catheter, biopsy needle, etc.), a medical imaging device providing real-time images for guidance during the IGT procedure, and possibly one or more patient monitoring devices; an oncology radiation therapy procedure employing a linear accelerator (LINAC) and associated control computer; a mammography procedure for breast cancer screening; and so forth.

As used herein, an “instance of a medical procedure” refers to the performance of the medical procedure on a specific patient (who may be an in-patient or an out-patient) to achieve the clinical purpose of the medical procedure with respect to that specific patient. While all instances of a given medical procedure generally follow the workflow of the medical procedure, given instances of the medical procedure may be different from one another in various ways. Such differences may be due to patient-specific considerations (for example, the workflow of an instance of the procedure may be modified to accommodate a patient who has a specific chronic medical condition, a specific disability, or is distinct in some other way such as being of unusually small/large stature or so forth). Differences between instances of the medical procedure may also arise from differences in the way medical personnel execute the workflow in a specific instance, for example using different order-of-operations, using different configuration settings for a medical device used in the instance, and/or so forth. Differences between instances can also arise based on which feature(s) of the medical device medical personnel utilize. Differences can also arise due to mistakes made by the medical personnel performing certain instances of the procedure. These are merely non-limiting examples, and other sources of differences between instances of a given medical procedure may be operative depending on the nature of the medical procedure, the configurability, features, and other aspects of the medical device(s) used, and so forth.

The following relates the generation of training videos or animations (or, more generally, educational content) for various medical imaging workflows. The goal is to create a training video or animation (or, more generally, educational content) of an example of best instance of a particular imaging procedure, as well as possibly also of one or more instances in which common mistakes occur during the imaging procedure.

One way to generate such content is to place a video camera in the imaging bay that captures the imaging device and the controller display. However, there are several problems with this approach. First, due to privacy concerns and simple practicalities, it is usually not possible to record every instance of a given imaging procedure. Hence, this approach requires the surgical staff to perform a re-enactment of the best instance of the imaging procedure. This is time consuming and impractical in many cases. Even if a re-enactment can be done, it may not perfectly replicate the successful example. Furthermore, clinicians are likely to be uncooperative in re-enacting examples of mistakes during procedures. As yet a further difficulty, in a complex procedure such as an Image Guided Therapy (IGT) procedure, there are multiple cooperating parts, e.g. in IGT there is the IGT imaging device and its controller, and the catheter and its controller.

The disclosed approaches do not compromise patient privacy in generating the educational content, and do not interfere with the medical procedure by deploying a video recorder or other recordation device in the medical theater to record the medical procedure. Rather, the disclosed approaches leverage the machine logs of the imaging device and any other ancillary hardware or software (e.g., the catheter in IGT) to create an animation for use in training. Such machine logs are automatically and non-obtrusively generated by many complex medical devices such as medical imaging devices, IGT systems, radiation therapy systems, and so forth, and record automatically generated data such as device sensor readings and operational metrics such as X-ray tube current and voltage, magnetic field gradient currents, and so forth. The machine logs are generated for the purpose of recording device operation for use in diagnosing device malfunctions, and for the purpose of providing data for predictive analyses in support of preventative maintenance. In embodiments disclosed herein, the machine logs are leveraged to provide information on actually performed instances of medical procedures. Other log data may also be generated. For example, medical imaging devices produce final clinical images that are typically stored together with relevant imaging device configuration, scan settings, operational parameters, and/or so forth in a Picture Archiving and Communication System (PACS). Radiation therapy devices store substantial information on instances of a radiation therapy treatment procedure, such as radiation exposure/dosage information, radiation beam configuration information, and so forth.

To leverage log data for use in generating educational content, as disclosed herein, the database(s) of log data are mined using a procedure quality analysis to identify best (and possibly worst) instances of instance of the workflow. This entails applying a first algorithm to identify instances of the imaging procedure, e.g. in a simple example based on a procedure identification field that is filled in by the imaging technician during exam setup. Then, one or more criteria are applied to identify the best/worst instances. (In an alternative approach, an instance or instances to be used for generating educational content may be manually identified by a medical department supervisor or other medical personnel with requisite expertise).

Using the log data for the identified (e.g.) best instance of the imaging procedure, a simulator is then applied to virtually re-create this best instance of the imaging procedure based at least in part on the log data for the identified instance. This may include a manual and/or automated first step in which the log data are pre-processed to remove or shorten unnecessary information (e.g. extraneous mouse clicks). Depending on the particulars of the imaging procedure being simulated, the simulator may include an image reconstruction engine and a database of simulated or suitably anonymized imaging data, a virtual reality (VR) representation of the three-dimensional (3D) working environment including the imaging device, catheter, et cetera), a simplified two-dimensional (2D) representation where appropriate, an imaging device controller simulator, and/or so forth. The (possibly pre-processed) log data are used to configure the simulation.

The animation is then generated by creating a video or time sequence of animation steps generated by the simulation. The animation may then be manually edited, for example to insert pauses for voice explanation overlays, to add visual, textual, and/or audio annotations, and/or so forth. The resulting training animation may optionally be converted to a video clip (e.g., stored as an MPEG file or in some other digital video format), or alternatively may be retained in an animation file format (e.g., an MB or MA Maya format or other animation file format such as FBX, 3DS, or so forth), and is then distributed for use in educational programs.

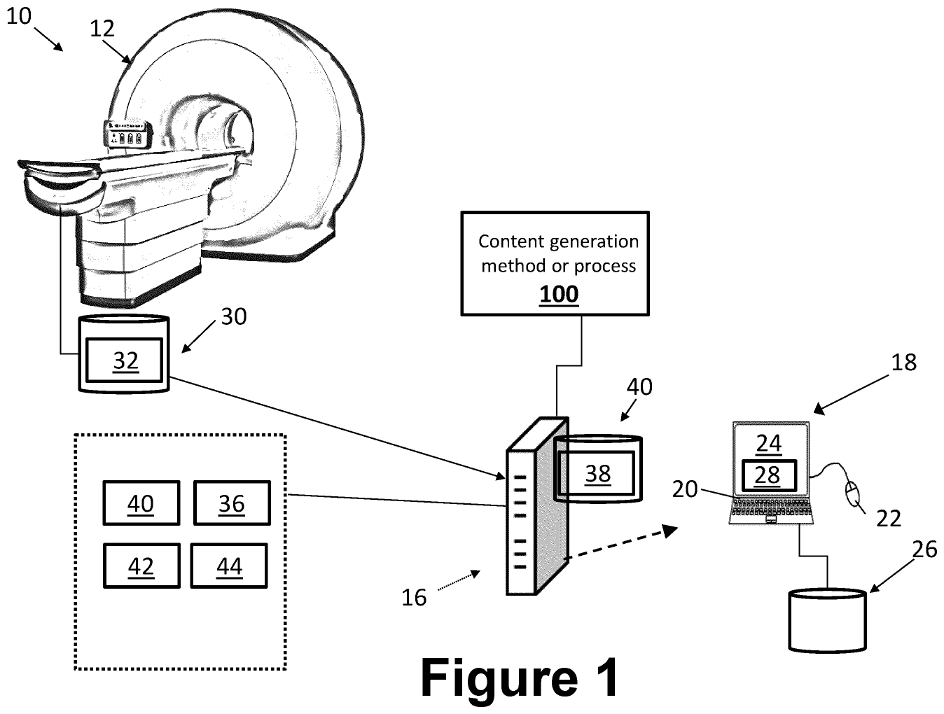

With reference to FIG. 1, an illustrative educational content generation system or apparatus 10 for generating educational content for a medical procedure employing one or more medical devices 12 (e.g., an illustrative medical imaging device 12; or a radiation therapy device; or a combination of the medical imaging device 12 and a biopsy needle, catheter, or other interventional instrument used cooperatively to perform an image guided therapy (IGT) procedure; or so forth). By way of some non-limiting illustrative examples, the medical imaging device 12 may be an interventional X-ray (IXR) or other interventional radiology (IR) system (used in combination with at least one interventional instrument in an IGT procedure), a magnetic resonance imaging (MRI) scanner, a computed tomography (CT) scanner, a positron emission tomography (PET) scanner, a gamma camera for performing single photon emission computed tomography (SPECT), or so forth. As shown in FIG. 1, the educational content generation system 10 includes, or is accessible by, a server computer 16 typically disposed remotely from the medical device(s) 12 used in the medical procedure for which content is to be generated.

The medical device 12 includes a non-transitory computer readable medium comprising a database 30 storing log data 32 related to operation of the device. The log data 32 are generated by the medical device(s) 12, for example by sensors of the medical imaging device, by a controller of the imaging device, and/or so forth. In one commonly used format, the sensors, controller, and so forth generate timestamped messages that form the log data 32. The database 30 can also store quality metrics and thresholds for procedures that can be identified from the log data 32. The database 30 may also comprise multiple databases—for example, the illustrative medical imaging device 12 may generate machine log data as just described that is stored in a machine log database (not shown), and may also generate imaging examination data including images and associated imaging device setting that are stored in a PACS database (not shown).

The server computer 16 comprises a computer or other programmable electronic device running software, such software implementing a monitor 36 in communication with the device 12 that analyzes the log data in the database 30 to identify log data for an educational instance of a procedure (i.e., medical examination) performed using the device 12. The log data 32 for the educational instance of the medical procedure can processed by the server computer 16 to generate an educational content unit 38 for training of technicians who operate the device 12. For example, the educational content unit 38 can comprise an animation, a video, and/or a series of images, showing a “best” instance of the procedure, or alternatively an instance of the procedure in which a mistake was made (i.e., to highlight the mistake in the procedure), or a “non-optimal” or “non-recommended usage” where there is a lower quality threshold that the logged procedure fell below. As used herein, the term “suboptimal performance” instance (and variants thereof) refers to an instance of the procedure in which a mistake was made, a non-optimal instance, or a non-recommended usage. In a common implementation, the server computer 16 may be a server computer owned or leased or otherwise under the control of the vendor of the medical device 12, and the log data (or portions thereof, such as the machine log) are uploaded from the database 30 to the vendor's server computer 16 on an occasional basis (e.g., daily). In this case, the analysis of the log data can be performed at the server computer 16 using the copy of the database content stored at the server computer 16.

The server computer 16 includes or is in communication with a non-transitory computer readable medium 40 storing instructions executable by the server computer 16 to perform a method or process 100 implemented by the educational support system 10 for generating the educational content units 38 for the device 12. In some examples, the method 100 may be performed at least in part by cloud processing (that is, the server computer 16 may be implemented as a cloud computing resource comprising an ad hoc network of server computers).

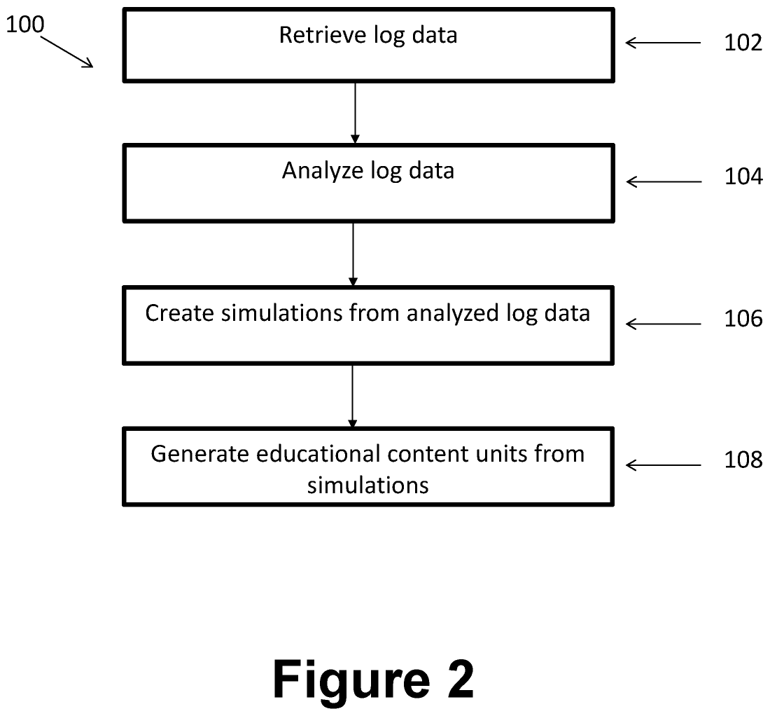

With reference to FIG. 2, and with continuing reference to FIG. 1, an illustrative embodiment of an instance of the method 100 is diagrammatically shown as a flowchart. At an operation 102, the server computer 16 retrieves the log data 32 of medical procedures performed using the device 12 from the database 30. (The operation 102 may be performed automatically, at least for machine log data, in the case where the server computer 16 is a vendor's server computer as part of the scheduled upload of the log data of the database 30 to the server computer 16, as previously noted. In this case, if additional data such as PACS database log data is needed to generate the educational content, then this PACS log data may be transferred per operation 102 as a separate transfer operation coordinated with the hospital or other entity controlling the PACS.) In addition, the operation 102 can include log data processing, for example the retrieved log data 32 can be sorted in appropriate groups (e.g., sorted by type of procedure, anatomy imaged, identification of technician, and so forth) using, for example, a rule-based algorithm or a machine-learning classification algorithm implemented by the server computer 16.

At an optional operation 104, the monitor 36 analyzes the log data 32 to identify an instance of a medical imaging procedure performed using the device 12. In one example embodiment, a procedure quality analysis is implemented by the monitor 36 on the log data 32 to identify the educational instance of the procedure. To do so, the procedure quality analysis includes applying one or more criteria to the log data 32 to identify the educational instance of the procedure as one of (i) an instance of the procedure which the quality analysis indicates exceeds a quality threshold (i.e., a “best” case) or (ii) an instance of the procedure that illustrates a mistake (i.e., a “worst” case). For example, the monitor 36 is configured to implement an algorithm to detect which procedure is being done by looking at steps taken during the configuration of the device, and usage steps to achieve a goal (e.g. configuring an exam card for the device 12 and subsequently running a knee scan on the device). The monitor 36 then implements a metric to assess the quality of the procedure being performed on the device 12 and thresholds to indicate when the usage is a representative candidate for good practice or bad practice examples. To do so, a probability of log files 32 belonging to each group sorted by the rule-based algorithm or the machine-learning classification algorithm is assigned to the group of log files, with the group with the highest probability being chosen for a simulation.

As noted, the operation 104 is optional. In an alternative embodiment, the educational instance of the medical procedure is identified manually. As an example of this, in the case where the server computer 16 is a vendor's server computer, a customer (e.g. a hospital) may contract with the vendor to produce educational content from an educational instance of the medical procedure that is identified by the customer. The customer may identify the educational instance of the medical procedure based on the expertise of customer (e.g. hospital) personnel who manually review records of instances of the medical procedure performed at the hospital to identify an instance that is rated to be of particular high quality. Conversely, the customer may identify the educational instance of the medical procedure as an instance that contains a certain mistake that the hospital has identified as a common mistake that is repeatedly being made at the hospital.

At an operation 106, the monitor 36 simulates or creates a simulation of the identified educational instance of the procedure using the identified log data for the educational instance of the procedure. In some cases, the log data 32 for the educational instance of the procedure can be pre-processed to remove unnecessary information. If the operation 104 is performed and identifies the educational instance as the group with the highest probability, then if the difference between the expected quality score and the actual one be small enough, the group is determined to be of high enough quality for educational material to be generated. (On the other hand, if a single instance of the medical procedure has been identified as the educational instance then this forms the “group of steps” from which the educational content is to be generated). This group of steps is then fed into a simulation environment run on the server computer 16 that mimics the device interface for the device 12 in question. In the simulation environment, the steps are taken in the same order as they were performed and recorded in the actually performed educational instance, as recorded in the log data for the educational instance. The simulator suitably includes user interface elements, automatically generates 3D animations from 3D models of the medical device(s) 12, for example a computer-aided design (CAD) representation of the medical device(s) 12. Additionally, the simulator may include an image reconstruction simulator for the case where the medical device(s) 12 includes a medical imaging device (e.g. MRI, CT, PET, et cetera) that generates images by reconstructing acquired imaging data. In the case of a radiation therapy (RT) procedure performed by a LINAC or other RT delivery device, the simulator may include mathematical models for computing radiation dose distributions and/or other relevant radiation output representations based on radiation delivery parameters employed in the educational instance of the RT procedure as recorded in the log data of the RT device. These are merely illustrative examples, and the components and implementation of the simulator depends on the medical device(s) 12 employed in the medical procedure for which educational content is being generated. Since some medical procedures may accidently result in poor results, outliers can be removed by observing the repeatability of the best/worst situations by initially withholding the replacement educational content for a period of time before overwriting the current best/worst.

At an operation 108, an educational content unit 38 is generated from the simulations. The simulations, and thereby the educational content units 38, can be created in a variety of manners.

In one example embodiment, the medical device(s) 12 comprise a medical imaging device, the medical procedure is a medical imaging procedure, and the identified log data 32 for the educational instance of the procedure include imaging data generated by the educational instance of the medical imaging procedure. In this embodiment, the creating operation 106 includes using a simulator comprising an image reconstruction engine 42 implemented in the server computer 16 to create the simulation including at least one reconstructed image by applying the image reconstruction engine to the imaging data generated by the educational instance of the medical imaging procedure. The content of the corresponding educational content unit 38 here includes the at least one reconstructed image.

In another example, the log data 32 can further include one or more settings of the image reconstruction engine 42 used in the educational instance of the procedure. The creating operation 106 then includes using the image reconstruction engine 42 to create the simulation to include the at least one reconstructed image generated by applying the image reconstruction engine with the one or more settings of the image reconstruction engine used in the educational instance of the procedure to the imaging data generated by the educational instance of the medical imaging procedure.

In a further example, the simulator can comprise an imaging device controller simulator 44 implemented in the server computer 16 to create the simulation comprising a simulated display of the imaging device controller during the educational instance of the medical imaging procedure. The content of the corresponding educational content unit 38 includes the simulated display of the imaging device controller.

In another example embodiment, the log data 32 include location data for the device 12 and/or mobile components of the device (e.g., if the device is an MRI imaging device, then locations of mobile components, such as local coils, can be simulated). Here the log data may only include an indication that a particular type of local coil was used in the educational instance of the medical procedure, but the location of that local coil can be inferred from the type of local coil, e.g. a head coil is inferred to be placed over the patient's head whereas a torso coil is inferred to be placed on or around the patient's torso or so forth. The patient location in the frame of reference of the medical imaging device is typically recorded in terms of the robotic patient support position readings. In this embodiment, the creating operation 106 includes using a three-dimensional (3D) virtual reality (VR) simulator run on the server computer 16 to generate a VR simulation video of the locations of the device 12 and/or components thereof during the educational instance of the medical imaging procedure. The content of the corresponding of the educational content unit 38 includes the VR simulation video.

To create the educational content unit(s) 38 at the generating operation 108, the server computer 16 is programmed to create a video in the simulation environment based on the chain of commands from the simulation. The operation 108 may optionally include providing a content editing user interface (UI) by which a user may add annotations (visual, audio, and/or textual, e.g. closed captions), delete unnecessary video segments, or otherwise edit the educational content to highlight important aspects of the instance of the medical procedure. For example, if the educational instance was selected because it includes a common mistake, the user may operate the content editing UI to add an annotation identifying the mistake and explaining why it is a mistake and how to avoid the mistake. The content editing UI is chosen based on the format of the educational content—for example, if the content format is video then the content editing UI may be a video editing UI; if the content format is an animation file format then the content editing UI may be an animation editing UI; if the content is a still image then the content editing UI may be an image editing UI; and/or so forth.

Referring back to FIG. 1, the educational content unit(s) 38 output by operation 108 may be consumed in various ways. In one illustrative example, the server computer 16 is in communication with an electronic processing device 18, such as a workstation computer located in at the customer (e.g. hospital). The illustrative workstation 18 includes typical components, such as an electronic processor 20 (e.g., a microprocessor), at least one user input device (e.g., a mouse, a keyboard, a trackball, and/or the like) 22, and a display device 24 (e.g. an LCD display, plasma display, cathode ray tube display, and/or so forth). In some embodiments, the display device 24 can be a separate component from the workstation 18 or may include two or more display devices. The electronic processor 20 is operatively connected with one or more non-transitory storage media 26. The non-transitory storage media 26 stores instructions executable by the at least one electronic processor 20 to present the educational content unit(s) 38 received from the server computer 16. For example, these instructions may implement a video player to present educational content comprising video content, and/or an image display program to present educational content comprising still images, and/or an animation presentation program to present educational content stored in an animation file format, and/or so forth. The instructions optionally include instructions to generate a graphical user interface (GUI) 28 for display on the display device 24 that coordinates the presentation of the educational content, especially if the educational content unit(s) 38 are multimedia content in the sense that the educational content is provided in multiple formats (e.g., video, still images, and animations). For example, the content-editing UI, the video editing UI, and/or the image editing UI, can be displayed on the GUI 28.

In most instances, the generating operation 108 includes generating a video representing the identified educational instance of the procedure from the created simulation; although other forms of media may be generated (e.g., images, content in an animation file format, content stored as a PowerPoint presentation, textual and graphical representations (e.g., flowcharts) and so forth). In other instances, a time sequence of animation steps can be created from the created simulation. The electronic processor 20 is programmed to implement video editor enabling a user to edit the video to include overlays, animations, or delete segments. Once created, the education content units 38 can be presented to the technicians.

The educational content units 38 can then be stored in the server computer 16 (and their associated metrics and links to associated data of the device 12) and/or at the non-transitory storage medium 26. The educational content generator may optionally also include a matching algorithm (not shown) implemented by the server computer 16 that suggests educational content units to users based on information on the users such as performance ratings. Hence, remedial content units may be suggested to users who do not exceed some performance rating threshold of good practice, or suggests content on avoidance of common mistakes to users who are new to the system or are below the lower threshold and exceed a certain frequency threshold (i.e. that error occurs frequently enough). Each best/worst case simulation is optionally overwritten (or archived) when new data generated in the field represents a closer/further result to the current best/worst values.

### Example

The following provides an example of the method 100. A technician performs a brain imaging procedure on a patient using a new MRI device (e.g., an Achieva 3T MR device). The technician has had time to experiment with the system. The steps taken during the imaging procedure were recorded in the database 30 and compiled into a procedure using the log data 32. Based on the steps taken, their order, and the parameters associated with each step, the imaging procedure is assigned a score. This score is an aggregate of multiple metrics such as expected image quality and relative signal-to-noise ratio.

Compared to the current threshold score associated with the procedure, the recorded procedure is performing well. As such, it is flagged for content generation to create the educational content unit 38. After checking to see that there is no previously generated content with similar conditions and a higher score the content generation begins.

The simulation environment for the MRI device with the same software version is started. An automated program is loaded with the set of commands and begins recording. The commands are executed in a slow and controlled manner and a screenshot is taken at each step. After the process is finished, the education content unit 38 video is annotated at each step and the screenshots are compiled in a document. This education content unit 38 is then used to supplement any existing generic educational content for the same system.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing:
a database storing log data automatically generated by one or more medical devices; and
instructions readable and executable by at least one electronic processor to perform a method for generating educational content units, the method comprising:
analyzing the log data contained in the database to identify log data for an educational instance of a procedure performed using the one or more medical devices;
creating a simulation of the identified educational instance of the procedure using the identified log data for the educational instance of the procedure; and
generating an educational content unit from the simulation.

2. The non-transitory computer readable medium of claim 1, wherein the analyzing includes:
implementing a procedure quality analysis on the log data contained in the database to identify the educational instance of the procedure.

3. The non-transitory computer readable medium of claim 2, wherein the analyzing includes:
applying one or more criteria to the log data to identify the educational instance of the procedure as one of (i) an instance of the procedure which the quality analysis indicates exceeds a quality threshold or (ii) an instance of the procedure that illustrates a suboptimal performance of the instance of the procedure.

4. The non-transitory computer readable medium of claim 1, wherein creating includes:
pre-processing the identified log data for the educational instance of the procedure to remove unnecessary information.

5. The non-transitory computer readable medium of claim 1, wherein the one or more medical devices comprise one or more medical imaging devices, the procedure is a medical imaging procedure, the identified log data for the educational instance of the procedure include imaging data generated by the educational instance of the medical imaging procedure, and the creating includes:
using a simulator comprising an image reconstruction engine to create the simulation comprising at least one reconstructed image by applying the image reconstruction engine to the imaging data generated by the educational instance of the medical imaging procedure;
wherein content of the educational content unit includes the at least one reconstructed image.

6. The non-transitory computer readable medium of claim 5, wherein the log data for the educational instance of the procedure further includes one or more settings of the image reconstruction engine used in the educational instance of the procedure, and the creating includes:
using the image reconstruction engine to create the simulation comprising the at least one reconstructed image generated by applying the image reconstruction engine with the one or more settings of the image reconstruction engine used in the educational instance of the procedure to the imaging data generated by the educational instance of the medical imaging procedure.

7. The non-transitory computer readable medium of claim 1, wherein the log data include location data for the one or more medical devices and/or mobile components of the one or more medical devices, and the creating includes:
using a three-dimensional (3D) virtual reality (VR) simulator process to generate a VR simulation video of the locations of the one or more medical devices and/or components thereof during the educational instance of the medical imaging procedure;
wherein content of the educational content unit includes the VR simulation video.

8. The non-transitory computer readable medium of claim 1, wherein the one or more medical devices comprise one or more medical imaging devices, the procedure is a medical imaging procedure, the log data include imaging device controller data generated by an imaging device controller of the one or more medical imaging devices, and the creating includes:
using a simulator comprising an imaging device controller simulator to create the simulation comprising a simulated display of the imaging device controller during the educational instance of the medical imaging procedure;
wherein content of the educational content unit includes the simulated display of the imaging device controller.

9. The non-transitory computer readable medium of claim 1, wherein the generating includes:
generating a video representing the identified educational instance of the procedure from the created simulation.

10. The non-transitory computer readable medium of claim 9, wherein the generating of the video includes:
generating a time sequence of animation steps from the created simulation.

11. The non-transitory computer readable medium of claim 9, wherein the generating of the video includes:
providing a video editor enabling a user to edit the video to include overlays, animations, or delete segments.

12. An apparatus, comprising:
a database storing log data automatically generated by one or more medical imaging devices;
at least one electronic processor; and
instructions readable and executable by the at least one electronic processor to perform a method for generating educational content units, the method comprising:
retrieving log data from the database for an instance of a medical imaging procedure performed using the one or more medical devices;
simulating the instance of the medical imaging procedure using the retrieved log data to generate a simulation of the instance of the medical imaging procedure; and
generating an educational content unit from the simulation of the instance of the medical imaging procedure.

13. The apparatus of claim 12, wherein the analyzing includes:
implementing a procedure quality analysis on the log data contained in the database to identify the educational instance of the procedure.

14. The apparatus of claim 13, wherein the analyzing includes:
applying one or more criteria to the log data to identify the educational instance of the procedure as one of (i) an instance of the procedure which the quality analysis indicates exceeds a quality threshold or (ii) an instance of the procedure that illustrates a suboptimal performance of the instance of the procedure.

15. The apparatus of claim 12, wherein the generating includes:
generating a video representing the identified educational instance of the procedure from the created simulation.

16. The apparatus of claim 12, wherein the method further includes:
analyzing the log data contained in the database to identify log data for an educational instance of a procedure performed using the one or more medical devices.

17. A method for generating educational content units, the method comprising:
retrieving log data from the database for an instance of a medical imaging procedure performed using the one or more medical devices;
analyzing the log data contained in the database to identify log data for an educational instance of a procedure performed using the one or more medical devices;
simulating the instance of the medical imaging procedure using the analyzed log data to generate a simulation of the instance of the medical imaging procedure; and
generating an educational content unit from the simulation of the instance of the medical imaging procedure.

18. The method of claim 17, wherein the analyzing includes:
implementing a procedure quality analysis on the log data contained in the database to identify the educational instance of the procedure by applying one or more criteria to the log data to identify the educational instance of the procedure as one of (i) an instance of the procedure which the quality analysis indicates exceeds a quality threshold or (ii) an instance of the procedure that illustrates a suboptimal performance of the instance of the procedure.

19. The method of claim 17, wherein the generating includes:
generating a video representing the identified educational instance of the procedure from the created simulation.

20. The method of claim 19, wherein the generating of the video includes:
generating a time sequence of animation steps from the created simulation.
